(19) United States
(12) Patent Application Publication
(10) Pub. No.: US 2010/0022851 A1
(43) Pub. Date: Jan. 28, 2010

(54) MEASUREMENT PROCESSING APPARATUS OF PHYSIOLOGY SIGNALS

(76) Inventor: Yang-Han Lee, Taipei (TW)

Correspondence Address: NORTH AMERICA INTELLECTUAL PROPERTY CORPORATION, P.O. BOX 506, MERRIFIELD, VA, 22116, US

(21) Appl. No.: 12/477,885

(22) Filed: Jun. 3, 2009

(30) Foreign Application Priority Data
Jul. 22, 2008 (TW) 097127812

(51) Int. Cl. A61B 5/00; H03M 1/12

(52) U.S. Cl. 600/301; 341/141

(57) ABSTRACT

A measurement processing apparatus is disclosed for processing plurality of physiology signals. The measurement processing apparatus includes a multiplex device, an analog-to-digital conversion module and a control signal generator. The multiplex device generates a multiplex signal composed of the physiology signals with output sequence controlled by a first control signal. Furthermore, the multiplex device dispenses each physiology signal with one corresponding multiplex density based on the first control signal. The analog-to-digital conversion module converts the multiplex signal into a digital multiplex signal based on at least one adjustable bias voltage under control of a second control signal. The control signal generator generates the first control signal based on the feature values of the physiology signals. Also, the control signal generator generates the second control signal based on the voltage swing ranges of the physiology signals. The second control signal is synchronized with the first control signal.

### BACKGROUND OF THE INVENTION

1. Field of the Invention

The present invention relates to a measurement processing apparatus of physiology signals, and more particularly, to a measurement processing apparatus for processing the physiology signals based on the feature values thereof.

2. Description of the Prior Art

Along with rapid development of the biomedical engineering, a variety of advanced physiology signal detection apparatuses have been put into research uninterruptedly. Furthermore, with the aid of integrated circuits for performing digital signal processing, the physiology signals can be measured and analyzed accurately. Nevertheless, since the physiology signals of a human body includes a blood oxygen signal, a blood pressure signal, an electrocardiograph (ECG) signal, an ocular pressure signal, a blood sugar signal, and other various physiology signals, and therefore a variety of physiology signal detection apparatuses are required for measuring different physiology signals. Normally, the original physiology signals generated by a physiology signal detection apparatus are analog physiology signals. For that reason, prior to performing digital signal processing for accurately analyzing the physiology signals, the analog physiology signals should be converted into digital physiology signals by an analog-to-digital converter.

In general, the analog-to-digital converter requires at least one conversion reference voltage, such as a high bias voltage or a low bias voltage, for performing an analog-to-digital conversion operation. However, the voltage swing ranges of different analog physiology signals may have a significant discrepancy. For instance, the voltage swing range of a blood pressure signal may reach a range of about several voltages, and the voltage swing range of a blood oxygen signal is limited to a range of only hundreds of milli-volts typically. In view of that, according to well-known prior art, two different analog-to-digital converters having different high/low bias voltages are required to perform analog-to-digital conversion operations on the blood pressure signal and the blood oxygen signal respectively, which results in a costly measurement processing apparatus and power saving operation of measuring processes is hard to be realized.

Besides, if the analyzing operations of physiology signals are performed by a single signal processor, a multiplex device is further required so as to perform a time-division multiplexing operation for dispensing each physiology signal with one multiplex time slot periodically. However, taking the blood pressure signal for instance, the blood pressure signal has an effective pressure range only between the systolic pressure and the diastolic pressure, and therefore the blood pressure signal having a pressure outside the effective pressure range is of no use and can be neglected. That is, while performing a measuring process for extracting the blood pressure signal, the blood pressure signal is not required to be taken until the blood pressure falls into the effective pressure range. On the other hand, the blood pressure signal is required to be taken continuously while the blood pressure falls into the effective pressure range. Accordingly, in the prior-art time-division multiplexing operation, parts of the dispensed multiplex time slots may be used to measure unwanted signal values. Furthermore, when a dedicated crucial interval is required for continuously fetching a certain physiology signal, parts of the desired data regarding the certain physiology signal may be lost in that the dispensed multiplex time slot thereof may not cover the dedicated crucial interval. In other aspect, since portable electronic products with multi-function integration have gained popularity, how to integrate a measurement processing apparatus of physiology signals into a portable electronic product, e.g. a mobile phone or a notebook computer, for easily measuring physiology signals by an end user has become an important top nowadays. Besides, due to the limited power capacity of a portable electronic product, how to devise a measurement processing apparatus having high-efficiency and power-saving operation mechanism for measuring a variety of physiology signals becomes another important topic.

### SUMMARY OF THE INVENTION

In accordance with an embodiment of the present invention, a measurement processing apparatus of physiology signals is disclosed for performing measurement operations on the physiology signals according to the feature values of the physiology signals. The measurement processing apparatus comprises a multiplex device and an analog-to-digital conversion module. The multiplex device is utilized for receiving the physiology signals and outputting a multiplex signal. Besides, the multiplex device has a functionality of dispensing each physiology signal with a corresponding multiplex density according to a first control signal. The first control signal is generated based on the feature values of the physiology signals. The analog-to-digital conversion module is electrically coupled to the multiplex device for receiving the multiplex signal. The analog-to-digital conversion module functions to convert the multiplex signal into a digital multiplex signal based on at least one bias voltage adjusted according to a second control signal in synchronization with the first control signal. The second control signal is generated based on the voltage swing ranges of the physiology signals.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a multiplex device for use in a measurement processing apparatus according to the present invention.

FIG. 2 is a schematic diagram showing a multiplex time slot allocation regarding the operation of the multiplex device shown in FIG. 1, having time along the abscissa.

FIG. 3 is a functional block diagram schematically showing a measurement processing apparatus of physiology signals in accordance with a first embodiment of the present invention.

FIG. 4 is a functional block diagram schematically showing a measurement processing apparatus of physiology signals in accordance with a second embodiment of the present invention.

FIG. 5 is a functional block diagram schematically showing a measurement processing apparatus of physiology signals in accordance with a third embodiment of the present invention.

### DETAILED DESCRIPTION

Hereinafter, preferred embodiments of the present invention will be described in detail with reference to the accompanying drawings. Here, it is to be noted that the present invention is not limited thereto.

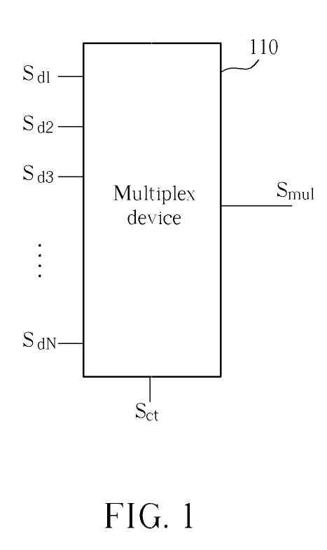

Please refer to FIG. 1, which is a multiplex device for use in a measurement processing apparatus according to the present invention. As shown in FIG. 1, the multiplex device 110 is employed to generate a multiplex signal Sₘᵤₗ by outputting a plurality of received physiology signals S_{d1}, S_{d2}, S_{d3}˜S_{dN} in a sequence under control of a control signal S_{ct}. The number N is an integer greater than 1. In one embodiment, the plurality of physiology signals S_{d1}˜S_{dN} are outputted sequentially in a preset time division multiplex cycle and the control signal Sct is employed to dispense each physiology signal with one multiplex time slot according to the feature values of the physiology signals S_{d1}˜S_{dN}. That is, each time division multiplex cycle is sectioned into a plurality of multiplex time slots and the length of each multiplex time slot is determined by the feature value of one corresponding physiology signal. In view of that, the multiplex signal Sₘᵤₗ includes the plurality of physiology signals S_{d1}˜S_{dN} sequentially outputted within each time division multiplex cycle.

In another embodiment, the multiplex device 110 is utilized for dispensing the physiology signals S_{d1}˜S_{dN} with a plurality of multiplex densities and each multiplex density is employed to control the output time density of one corresponding physiology signal. A higher multiplex density indicates that the output time density of the corresponding physiology signal is also higher. Each multiplex density is determined by the feature value of one corresponding physiology signal. For that reason, the aforementioned time division multiplex cycle is not required in the embodiment based on the multiplex densities. For instance, when the multiplex density of the physiology signal S_{d1} is null, i.e. the physiology signal S_{d1} is currently of no use and can be neglected, the output of the physiology signal S_{d1} is prohibited at the moment so as to prevent fetching unwanted signal values. Alternatively, when the multiplex density of the physiology signal S_{d3} is full-scale, i.e. within the dedicated crucial interval corresponding to the physiology signal S_{d3}, the signal values of the physiology signal S_{d3} are continuously fetched during the dedicated crucial interval so as to prevent losing important signal values provided that no multiplex density of other physiology signal is full-scale. However, when a plurality of multiplex densities corresponding to different physiology signals are full-scale simultaneously, the output time densities of the physiology signals having full-scale multiplex density can be assigned the same value for equally sharing output time. Alternatively, each physiology signal can be assigned a priority, and the output time densities of the physiology signals having full-scale multiplex density are determined based on relative proportional relationship of the priorities corresponding to the physiology signals having full-scale multiplex density.

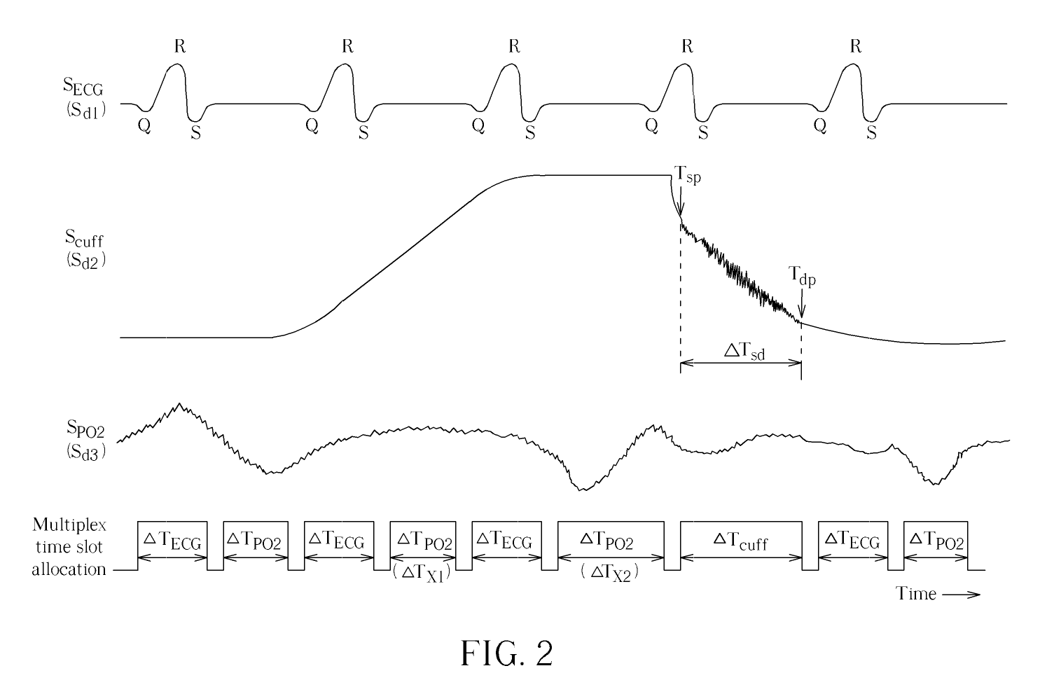

Please refer to FIG. 2, which is a schematic diagram showing a multiplex time slot allocation regarding the operation of the multiplex device 110 shown in FIG. 1, having time along the abscissa. In FIG. 2, ΔT_{ECG} represents the multiplex time slot dispensed for fetching an ECG signal S_{ECG}, ΔT_{cuff} represents the multiplex time slot dispensed for fetching a blood pressure signal S_{cuff}, and ΔT_{PO2} represents the multiplex time slot dispensed for fetching a blood oxygen signal S_{PO2}. The ECG signal S_{ECG}, the blood pressure signal S_{cuff} and the blood oxygen signal S_{PO2} are respectively corresponding to the physiology signals S_{d1}, S_{d2} and S_{d3} in FIG. 1. The feature value QRS of the ECG signal S_{ECG} is employed to define a QRS envelop, and the information related to cardiac operation is essentially provided based on the QRS envelop. That is, only the signal values of the ECG signal S_{ECG} corresponding to the QRS envelop are effective and required to be fetched. Since the QRS envelop is occurring periodically, the difference of successive QRS envelops occurring within a short time is normally not significant. For that reason, the multiplex density or the multiplex time slot corresponding to the ECG signal S_{ECG} can be assigned higher or longer during the interval of each QRS envelop. However, the multiplex density corresponding to the ECG signal S_{ECG} is unnecessary to be assigned full-scale, and each multiplex time slot corresponding to the ECG signal S_{ECG} is assigned just to cover one corresponding QRS envelop.

Since the feature values of the blood pressure signal S_{cuff} are the systolic pressure and the diastolic pressure, the signal values of the blood pressure signal S_{cuff} to be taken are suppose to focus on the fluctuation values within an effective pressure range between the systolic pressure and the diastolic pressure. In view of that, the signal values of the blood pressure signal S_{cuff} to be taken are during an interval between Tsp and Tdp shown in FIG. 2. Accordingly, the fluctuation values between Tsp and Tdp can be defined as effective pressure values, and the interval between Tsp and Tdp can be defined as a dedicated crucial interval ΔTsd corresponding to the blood pressure signal S_{cuff}. On the other hand, the signal values of the blood pressure signal S_{cuff} occurring external to the dedicated crucial interval ΔTsd is of no use and can be neglected. For that reason, the multiplex density of the blood pressure signal S_{cuff} external to the dedicated crucial interval ΔTsd can be assigned null and the multiplex density of the blood pressure signal S_{cuff} within the dedicated crucial interval ΔTsd can be assigned full-scale. Or otherwise, the multiplex time slot of the blood pressure signal S_{cuff} can be assigned the dedicated crucial interval ΔTsd extended from Tsp continuously to Tdp. In another embodiment, during the dedicated crucial interval ΔTsd, the multiplex density or the multiplex time slot corresponding to the blood pressure signal S_{cuff} is assigned higher or longer rather than full-scale or the whole interval ΔTsd, i.e. certain preset portion of output time density or multiplex time slot is still reserved for fetching other physiology signals during the interval ΔTsd.

The blood oxygen signal S_{PO2} is basically not a periodical signal, i.e. no dedicated crucial interval can be clearly defined for identifying important signal portion. Nevertheless, the signal values of the blood oxygen signal S_{PO2} having high variation rate can be defined to be the feature values thereof. Consequently, the multiplex density or the multiplex time slot of the blood oxygen signal S_{PO2} can be assigned higher or longer in the interval during which the blood oxygen signal S_{PO2} has high variation rate. On the contrary, the multiplex density or the multiplex time slot of the blood oxygen signal S_{PO2} can be assigned lower or shorter in the interval during which the blood oxygen signal S_{PO2} has low variation rate. Besides, in certain interval during which the variation rate of the blood oxygen signal S_{PO2} is tiny, the multiplex density of the blood oxygen signal S_{PO2} can be assigned null or no multiplex time slot is dispensed for fetching the blood oxygen signal S_{PO2}.

Please continue referring to FIG. 2, the multiplex time slot allocation indicates that the dedicated crucial interval ΔTsd is almost wholly assigned to be the dedicated multiplex time slot ΔT_{cuff} dispensed for fetching the blood pressure signal S_{cuff} and no multiplex time slot ΔT_{cuff} is assigned for fetching the blood pressure signal S_{cuff} during other intervals external to the dedicated crucial interval ΔTsd. Similarly, the intervals of the QRS envelops are almost assigned to be the multiplex time slots ΔT_{ECG} dispensed for fetching the ECG signal S_{ECG} and no multiplex time slot ΔT_{ECG} is assigned for fetching the ECG signal S_{ECG} during other intervals external to the QRS envelops. In view of that, the multiplex time slots ΔT_{ECG} are almost allocated periodically except for the dedicated crucial interval of other physiology signal such as the dedicated crucial interval ΔTsd of the blood pressure signal S_{cuff}. The length of the multiplex time slot ΔT_{PO2} dispensed for fetching the blood oxygen signal S_{PO2} is corresponding to the variation rate of the blood oxygen signal S_{PO2}. For instance, the multiplex time slot ΔT_{X1} is shorter following the low variation rate of the blood oxygen signal S_{PO2} around the multiplex time slot ΔT_{X1}, and the multiplex time slot ΔT_{X2} is longer following the high variation rate of the blood oxygen signal S_{PO2} around the multiplex time slot ΔT_{X2}.

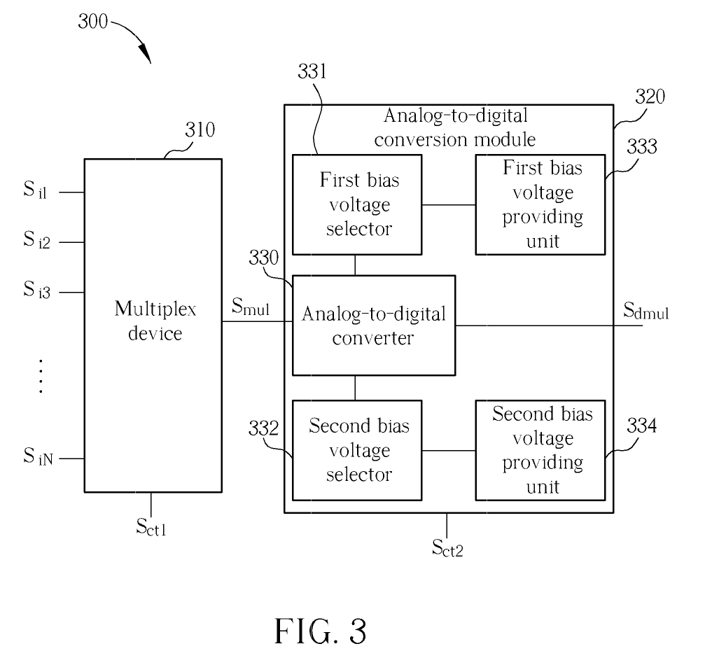

Please refer to FIG. 3, which is a functional block diagram schematically showing a measurement processing apparatus of physiology signals in accordance with a first embodiment of the present invention. As shown in FIG. 3, the measurement processing apparatus 300 comprises a multiplex device 310 and an analog-to-digital conversion module 320. The multiplex device 310 is employed to generate a multiplex signal Sₘᵤₗ by outputting a plurality of received physiology signals Sᵢ₁, Sᵢ₂, Sᵢ₃˜S_{iN} in a sequence under control of a first control signal S_{ct1}. The operational functionality of the multiplex device 310 is identical to that of the multiplex device 110 shown in FIG. 1, and for the sake of brevity, further discussion thereof is omitted. The analog-to-digital conversion module 320 comprises an analog-to-digital converter 330, a first bias voltage selector 331, a first bias voltage providing unit 333, a second bias voltage selector 332, and a second bias voltage providing unit 334.

The first bias voltage providing unit 333 functions to provide a plurality of first bias voltages. The first bias voltage selector 331 is employed to select a desirable first bias voltage out of the first bias voltages provided by the first bias voltage providing unit 333 according to a second control signal S_{ct2}. The selected first bias voltage is then furnished to the analog-to-digital converter 330. The second bias voltage providing unit 334 functions to provide a plurality of second bias voltages. The second bias voltage selector 332 is employed to select a desirable second bias voltage out of the second bias voltages provided by the second bias voltage providing unit 334 according to the second control signal S_{ct2}. The selected second bias voltage is then furnished to the analog-to-digital converter 330. The analog-to digital converter 330 is used to perform an analog-to-digital conversion operation on the multiplex signal Sₘᵤₗ for generating a digital multiplex signal S_{dmul}.

Since the voltage swing ranges of received physiology signals Sᵢ₁, Sᵢ₂, Sᵢ₃˜S_{iN} may have a significant discrepancy, the second control signal S_{ct2} is then employed to select suitable first and second bias voltages, functioning as high and low reference voltages required for performing an analog-to-digital conversion operation, based on the voltage swing ranges of different physiology signals Sᵢ₁, Sᵢ₂, Sᵢ₃˜S_{iN} received. For instance, while performing an analog-to-digital conversion operation on a blood pressure signal, the second control signal S_{ct2} is employed to select corresponding first and second bias voltages based on the voltage swing range of about several voltages corresponding to the blood pressure signal. Alternatively, while performing an analog-to-digital conversion operation on a blood oxygen signal, the second control signal S_{ct2} is employed to select corresponding first and second bias voltages based on the voltage swing range of about hundreds of milli-voltages corresponding to the blood oxygen signal.

In summary, although the measurement processing apparatus 300 of the present invention has a functionality of performing analog-to-digital conversion operations on different physiology signals, only one analog-to-digital converter, i.e. the analog-to-digital converter 330, is required for performing analog-to-digital conversion operations on different physiology signals. That is, in comparison with the prior art, the production cost of the measurement processing apparatus 300 can be reduced significantly; and furthermore, the power consumption regarding the operation of the measurement processing apparatus 300 can be lowered accordingly. Besides, the signal fetching operation of the multiplex device 310 is much more flexible based on the multiplex densities or the multiplex time slots determined by the feature values of different physiology signals, for achieving a high efficiency process of fetching the physiology signals. In view of the aforementioned, the measurement processing apparatus 300 having simplified architecture and low operation power consumption is especially suitable to be embedded in portable electronic products such as mobile phones, personal digital assistants (PDAs), notebook computers, or pocket personal computers.

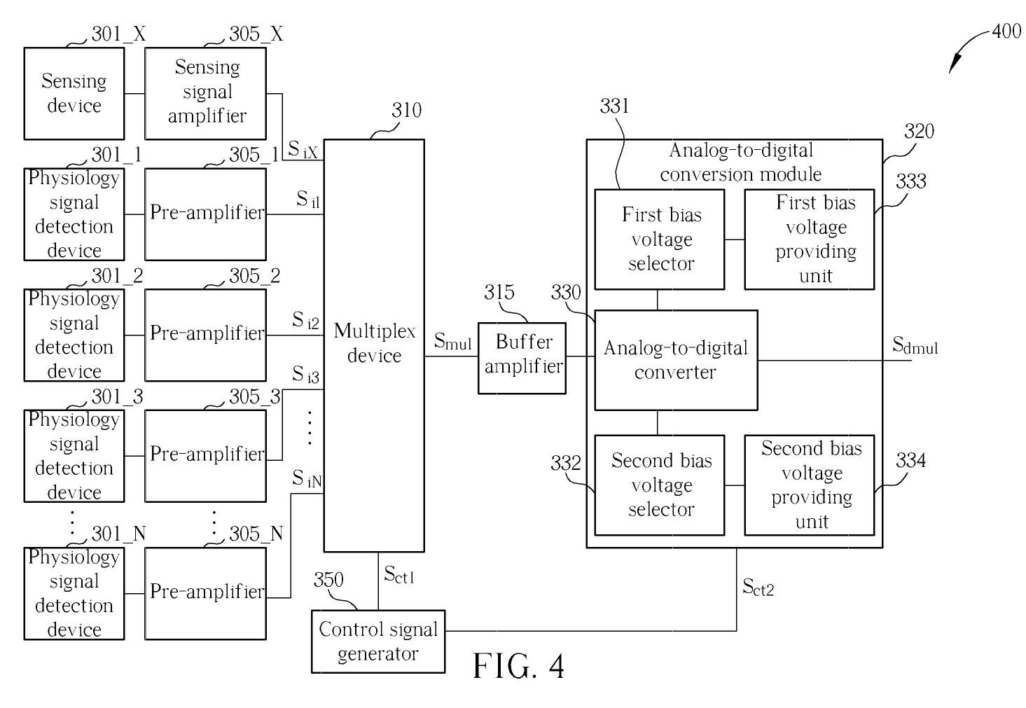

Please refer to FIG. 4, which is a functional block diagram schematically showing a measurement processing apparatus of physiology signals in accordance with a second embodiment of the present invention. As shown in FIG. 4, the measurement processing apparatus 400 is similar to the measurement processing apparatus 300 shown in FIG. 3, differing in that the measurement processing apparatus 400 further comprises a plurality of per-amplifiers 305_1˜305_N, a plurality of physiology signal detection devices 301_1˜301_N, a buffer amplifier 315, and a control signal generator 350. The multiplex device 310 and the analog-to-digital conversion module 320 shown in FIG. 4 have the same functionalities as aforementioned.

The physiology signal detection devices 301_1˜301_N are employed to perform various physiology detection processes for generating a plurality of original physiology signals. For instance, the physiology signal detection device 301_1 can be a blood oxygen signal detector for performing a blood oxygen signal detection process so as to generate an original blood oxygen signal; the physiology signal detection device 301_2 can be an ocular pressure signal detector for performing an ocular pressure signal detection process so as to generate an original ocular pressure signal; and the physiology signal detection device 301_3 can be a blood pressure signal detector for performing a blood pressure signal detection process so as to generate an original blood pressure signal. The pre-amplifiers 305_1˜305_N, respectively coupled between the physiology signal detection devices 301_1˜301_N and the multiplex device 310, are employed to perform signal amplification operations on the original physiology signals for generating a plurality of physiology signals Sᵢ₁, Sᵢ₂, Sᵢ₃˜S_{iN}. The dedicated signal amplification factor of each pre-amplifier is determined based on the voltage swing range of corresponding original physiology signal. For instance, the pre-amplifier for amplifying the blood pressure signal can be set to have a low signal amplification factor, and the pre-amplifier for amplifying the blood oxygen signal can be set to have a high signal amplification factor. The buffer amplifier 315 functions to perform a signal amplification operation on the multiplex signal Sₘᵤₗ or to enhance the driving ability of the multiplex signal Sₘᵤₗ. The control signal generator 350 is utilized for generating the first control signal S_{ct1} and the second control signal S_{ct2} according to the feature values of the physiology signals Sᵢ₁˜S_{iN}. The second control signal S_{ct2} is synchronized with the first control signal S_{ct1}. Accordingly, while the physiology signal Sᵢ₁, is forwarded by the multiplex device 310 under control of the first control signal S_{ct1}, the analog-to-digital conversion module 320 is controlled by the second control signal S_{ct2} in synchronization with the first control signal S_{ct1} so that the first bias voltage selector 331 and the second bias voltage selector 332 simultaneously select first and second bias voltages, corresponding to the physiology signal Si, respectively provided by the first bias voltage providing unit 333 and the second bias voltage providing unit 334. In view of that, the analog-to-digital converter 330 is then able to perform an optimal analog-to-digital conversion operation on the physiology signal Sᵢ₁ based on the selected first and second bias voltages.

In addition, as shown in FIG. 4, the measurement processing apparatus 400 may further comprise a sensing device 301_X and a sensing signal amplifier 305_X. The sensing device 301_X is employed to detect some non-physiology signal for generating an original sensing signal. The sensing signal amplifier 305_X performs a signal amplification operation on the original sensing signal for generating a sensing signal S_{iX}. The sensing device 301_X can be an image sensing device and the sensing signal S_{iX} is then an image sensing signal. Consequently, the first control signal S_{ct1} is further employed to dispense extra multiplex density or multiplex time slot based on the feature value of the sensing signal S_{iX}. Also, the second control signal S_{ct2} is further employed to select first and second bias voltages according to the voltage swing range of the sensing signal S_{iX} so that the analog-to-digital converter 330 is capable of performing an optimal analog-to-digital conversion operation on the sensing signal S_{iX}.

Similarly, compared with the prior art, the measurement processing apparatus 400 of the present invention makes use of only the analog-to-digital converter 330 for performing various analog-to-digital conversion operations, and therefore both the production cost and the operation power consumption can be reduced significantly. Besides, the signal fetching operation of the physiology signals and/or the non-physiology signal is much more flexible based on the multiplex densities or the multiplex time slots determined by the feature values of different physiology signals and/or the non-physiology signal, for achieving a high efficiency process of fetching the physiology signals and/or the non-physiology signal. In view of the aforementioned, the measurement processing apparatus 400 having simplified architecture and low operation power consumption is suitable to be embedded in portable electronic products such as mobile phones, personal digital assistants, notebook computers, or pocket personal computers.

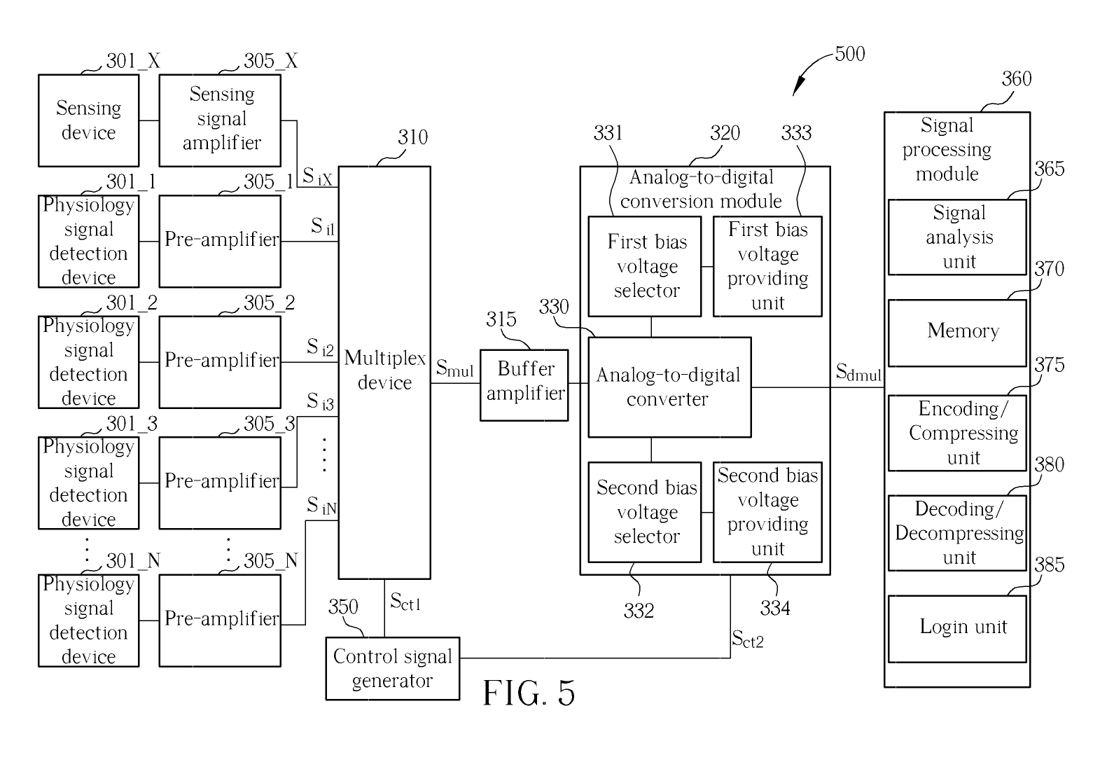

Please refer to FIG. 5, which is a functional block diagram schematically showing a measurement processing apparatus of physiology signals in accordance with a third embodiment of the present invention. As shown in FIG. 5, the measurement processing apparatus 500 is similar to the measurement processing apparatus 400 shown in FIG. 4, differing in that the measurement processing apparatus 500 further comprises a signal processing module 360 coupled to the analog-to-digital conversion module 320 for receiving the digital multiplex signal S_{dmul}. Except for the signal processing module 360, the other devices of the measurement processing apparatus 500 have the same functionalities as aforementioned. The signal processing module 360 comprises a signal analysis unit 365, a memory 370, an encoding/compressing unit 375, a decoding/decompressing unit 380, and a login unit 385. The signal analysis unit 365 functions to perform related signal analysis operations on the digital multiplex signal S_{dmul}. For instance, the signal analysis unit 365 may perform an analysis on the systolic and diastolic pressures of the blood pressure signal for determining whether the phenomenon of hypertension or hypotension occurs. Alternatively, the signal analysis unit 365 may perform an analysis on the ECG signal for determining whether the phenomenon of arrhythmia occurs.

The encoding/compressing unit 375 is used to perform an encoding/compressing operation on the digital multiplex signal S_{dmul} for generating an encoded/compressed signal so as to reduce required storage space and provide data encryption functionality. The memory 370 is utilized for storing the encoded/compressed signal or for directly storing the digital multiplex signal S_{dmul}. The memory 370 is a nonvolatile memory such as an electrically erasable programmable read only memory (EEPROM) or a flash memory. The decoding/decompressing unit 380 functions to perform a decoding/decompressing operation on the encoded/compressed signal so as to regain the digital multiplex signal S_{dmul}. The login unit 385 is employed to determine a client authority regarding the operations of the encoding/compressing unit 375 and the decoding/decompressing unit 380 according to a preset security rule. For instance, if the login data, inputted to the login unit 385 by a client, conforms to the preset security rule, the client is allowed to operate the encoding/compressing unit 375 and the decoding/decompressing unit 380 for performing related encoding/compressing or encoding/compressing operations. Furthermore, the authority may be graded, i.e. some client having high authority grade can be authorized to decode/decompress all the data stored in the memory 370 while the other client having low authority grade is authorized to decode/decompress only limited data stored in the memory 370.

Similarly, compared with the prior art, the measurement processing apparatus 500 of the present invention makes use of only the analog-to-digital converter 330 for performing various analog-to-digital conversion operations, and therefore both the production cost and the operation power consumption can be reduced significantly. Besides, the signal fetching operation of the physiology signals and/or the non-physiology signal is much more flexible based on the multiplex densities or the multiplex time slots determined by the feature values of different physiology signals and/or the non-physiology signal, for achieving a high efficiency process of fetching the physiology signals and/or the non-physiology signal. In addition, the measurement processing apparatus 500 further provides a security mechanism for protecting measurement data stored and a compressing/decompressing mechanism for reducing required storage space. In conclusion, the measurement processing apparatus 500 having simplified architecture and low operation power consumption is suitable to be embedded in portable electronic products such as mobile phones, personal digital assistants, notebook computers, or pocket personal computers.

The present invention is by no means limited to the embodiments as described above by referring to the accompanying drawings, which may be modified and altered in a variety of different ways without departing from the scope of the present invention. Thus, it should be understood by those skilled in the art that various modifications, combinations, sub-combinations and alternations might occur depending on design requirements and other factors insofar as they are within the scope of the appended claims or the equivalents thereof.

What is claimed is:

1. A measurement processing apparatus of physiology signals, comprising:
a multiplex device for receiving a plurality of physiology signals and outputting a multiplex signal, the multiplex device dispensing each of the physiology signals with a corresponding multiplex density according to a first control signal; and
an analog-to-digital conversion module, electrically coupled to the multiplex device for receiving the multiplex signal;
wherein the first control signal is generated based on feature values of the physiology signals.

2. The measurement processing apparatus of claim 1, further comprising:
a plurality of physiology signal detection devices, electrically coupled to the multiplex device, each of the physiology signal detection devices performing a corresponding physiology detection process so as to generate a corresponding physiology signal of the physiology signals.

3. The measurement processing apparatus of claim 2, further comprising:
a plurality of pre-amplifiers, each of the pre-amplifier being electrically coupled between the multiplex device and a corresponding physiology signal detection device of the physiology signal detection devices, for performing a signal amplification operation on a corresponding physiology signal of the physiology signals.

4. The measurement processing apparatus of claim 1, wherein the analog-to-digital conversion module is utilized for converting the multiplex signal into a digital multiplex signal based on at least one bias voltage adjusted according to a second control signal in synchronization with the first control signal;
wherein the second control signal is generated based on a plurality of voltage swing ranges of the physiology signals.

5. The measurement processing apparatus of claim 4, wherein the analog-to-digital conversion module comprises:
a first bias voltage providing unit for providing a plurality of first bias voltages;
a first bias voltage selector, electrically coupled to the first bias voltage providing unit, for selecting a corresponding first bias voltage out of the first bias voltages according to the second control signal; and
an analog-to-digital converter, electrically coupled to the multiplex device and the first bias voltage selector for receiving the multiplex signal and the corresponding first bias voltage respectively, for converting the multiplex signal into the digital multiplex signal based on the corresponding first bias voltage.

6. The measurement processing apparatus of claim 5, wherein the analog-to-digital conversion module further comprises:
a second bias voltage providing unit for providing a plurality of second bias voltages; and
a second bias voltage selector, electrically coupled between the second bias voltage providing unit and the analog-to-digital converter, for selecting a corresponding second bias voltage out of the second bias voltages according to the second control signal, the corresponding second bias voltage being forwarded to the analog-to-digital converter;
wherein the analog-to-digital converter converts the multiplex signal into the digital multiplex signal based on the corresponding first bias voltage and the corresponding second bias voltage.

7. The measurement processing apparatus of claim 4, further comprising:
a buffer amplifier, electrically coupled between the multiplex device and the analog-to-digital conversion module, for performing a signal amplification operation on the multiplex signal or for enhancing a driving ability of the multiplex signal.

8. The measurement processing apparatus of claim 4, wherein the multiplex device further receives a sensing signal, and wherein the measurement processing apparatus further comprises:
a sensing device, electrically coupled to the multiplex device, for performing a sensing process to generate the sensing signal;
wherein the multiplex device further dispenses the sensing signal with a multiplex density according to the first control signal, the first control signal is generated based on the feature values of the physiology signals and a feature value of the sensing signal, and the second control signal is generated based on the voltage swing ranges of the physiology signals and a voltage swing range of the sensing signal.

9. The measurement processing apparatus of claim 8, further comprising:
a sensing signal amplifier, electrically coupled between the multiplex device and the sensing device, for performing a signal amplification operation on the sensing signal.

10. The measurement processing apparatus of claim 8, wherein the sensing device is an image sensing device and the sensing signal is an image sensing signal.

11. The measurement processing apparatus of claim 8, further comprising:
a control signal generator, electrically coupled to the multiplex device and the analog-to-digital conversion module, for generating the first control signal based on the feature values of the physiology signals and the feature value of the sensing signal and for generating the second control signal based on the voltage swing ranges of the physiology signals and the voltage swing range of the sensing signal.

12. The measurement processing apparatus of claim 4, further comprising:
a control signal generator, electrically coupled to the multiplex device and the analog-to-digital conversion module, for generating the first control signal based on the feature values of the physiology signals and for generating the second control signal based on the voltage swing ranges of the physiology signals.

13. The measurement processing apparatus of claim 4, further comprising:
a signal processing module, electrically coupled to the analog-to-digital conversion module, for performing a signal analysis operation on the digital multiplex signal.

14. The measurement processing apparatus of claim 13, wherein the signal processing module comprises:
a signal analysis unit for performing the signal analysis operation on the digital multiplex signal; and
a memory for storing the digital multiplex signal.

15. The measurement processing apparatus of claim 13, wherein the signal processing module comprises:
a signal analysis unit for performing the signal analysis operation on the digital multiplex signal;
an encoding/compressing unit for performing an encoding/compressing operation on the digital multiplex signal to generate an encoded/compressed signal;
a decoding/decompressing unit for performing an decoding/decompressing operation on the encoded/compressed signal to regain the digital multiplex signal; and
a memory for storing the encoded/compressed signal.

16. The measurement processing apparatus of claim 15, wherein the signal processing module comprises:
a login unit for determining an authority grade of a client regarding operations of the encoding/compressing unit and the decoding/decompressing unit according to a security rule.

17. The measurement processing apparatus of claim 16, wherein if login data, inputted to the login unit by the client, conforms to the security rule, the client is allowed to operate the encoding/compressing unit and the decoding/decompressing unit for performing the encoding/compressing operation on the digital multiplex signal or for performing the decoding/decompressing operation on the encoded/compressed signal.

18. The measurement processing apparatus of claim 1, wherein the measurement processing apparatus is embedded in a portable electronic product.

19. The measurement processing apparatus of claim 18, wherein the portable electronic product is a mobile phone, a personal digital assistant (PDA), or a portable computer.

20. The measurement processing apparatus of claim 19, wherein the portable computer is a notebook computer or a pocket personal computer.
